(19) United States
(12) Patent Application Publication
(10) Pub. No.: US 2025/0170201 A1
(43) Pub. Date: May 29, 2025

(54) ONION EXTRACT, THE PREPARATION THEREOF AND THE USE FOR PROMOTING HAIR GROWTH THEREOF

(71) Applicant: FORMOSA BIOMEDICAL TECHNOLOGY CORPORATION, Taipei City (TW)

(72) Inventors: RUEY YU WANG, Taipei City (TW); TSONG-LONG HWANG, Taoyuan City (TW); YU-CHIA CHANG, Taoyuan City (TW); PEI LING WU, Changhua County (TW); WEI CHI CHEN, Changhua County (TW)

(73) Assignee: FORMOSA BIOMEDICAL TECHNOLOGY CORPORATION, Taipei City (TW)

(21) Appl. No.: 18/519,093

(22) Filed: Nov. 27, 2023

(51) Int. Cl. A61K 36/88; A61P 17/14

(52) U.S. Cl. CPC A61K 36/88; A61P 17/14; A61K 2236/331; A61K 2236/37; A61K 2236/55

(57) ABSTRACT

Onion extract, the preparation method thereof and the use for promoting hair growth thereof are provided, the preparation method comprises extracting an onion sample by subcritical water extraction, then obtaining an onion extract, wherein the extract temperature is 120° C.˜200° C.; the extract pressure is 1300˜1700 Psi; and the extract time is 5˜45 minutes.

### FIELD OF THE INVENTION

The present invention relates to a plant extract, its preparation method and uses thereof, in particular to an onion extract, its preparation method and its use for promoting hair growth.

### BACKGROUND OF THE INVENTION

Alopecia can be categorized into different patterns of hair loss, including diffuse, specific pattern, and localized. Additionally, it can be further classified based on damage to hair follicle cells into two types: scarring and non-scarring.

Scarring alopecia typically refers to pathological changes or necrosis of scalp hair follicles due to injury or disease, resulting in permanent scars. Common causes include infection, trauma, or illness. Non scarring alopecia, on the other hand, refers to hair follicles whose regenerative centers remain intact, retaining the ability to produce new hair. Common types of non scarring alopecia include male pattern baldness, alopecia areata, telogen effluvium, anagen effluvium, secondary syphilis, trichotillomania, and more.

In the past, folk remedies suggested that applying onion or ginger to the scalp, or creating a juice from onions or ginger to apply to the scalp, could stimulate hair follicles and promote hair growth. However, these folk remedies have been scientifically proven to lack a valid basis. If not properly controlled for concentration, they may even lead to scalp dermatitis, or redness and swelling on the scalp.

Alopecia has become a prevalent issue affecting both men and women in today's society. As a result, numerous products are available on the market for treating or preventing hair loss. However, there are still limited options for non chemical products. Recognizing this, the Applicant has actively researched natural products that promote hair growth.

### SUMMARY OF THE INVENTION

The present invention provides a method for preparing an onion extract, comprising: step (1): providing an onion sample, and step (2): subjecting said onion sample to extraction using a subcritical water extraction method, with an extraction temperature ranging from 120° C. to 200° C., an extraction pressure ranging from 1300 to 1700 Psi, and an extraction time ranging from 5 to 45 minutes, resulting in an onion extract.

The present invention also provides an onion extract prepared by the method comprises step (1): providing an onion sample, and step (2): subjecting said onion sample to extraction using a subcritical water extraction method, with an extraction temperature ranging from 120° C. to 200° C., an extraction pressure ranging from 1300 to 1700 Psi, and an extraction time ranging from 5 to 45 minutes, resulting in an onion extract.

The present invention further provides a method for promoting hair growth, comprising administering the onion extract to a subject in need thereof.

### BRIEF DESCRIPTION OF THE DRAWINGS

FIG. 1 depicts various extracts obtained from three different onion varieties using different extraction methods, including high-pressure organic solvent extraction, subcritical water extraction, reflux extraction, and fresh onion juice extraction. The impact of these extracts on the proliferation of human hair dermal papilla cells (HHDPC) is evaluated. FIG. 1a represents purple onions (POnion); FIG. 1b represents yellow onions (YOnion); FIG. 1c represents white onions (WOnion). Water (H2O) and dimethyl sulfoxide (DMSO) serve as control groups. Asterisks (*) indicate significant differences; *: p<0.5; **: p<0.05; ***: p<0.01.

FIG. 2 illustrates the HPLC fractionation of 200° C. subcritical water extracts from the three onion varieties based on signal intensity. FIG. 2a represents purple onions (POnion); FIG. 2b represents yellow onions (YOnion); FIG. 2c represents white onions (WOnion).

FIG. 3 shows effects of different HPLC fractions (based on signal intensity) of 200° C. subcritical water extracts from three onion varieties on the proliferation of human hair dermal papilla cells (HHDPC). FIG. 3a represents purple onions (POnion); FIG. 3b represents yellow onions (YOnion); FIG. 3c represents white onions (WOnion). Water (H2O) and dimethyl sulfoxide (DMSO) serve as control groups. Asterisks (*) indicate significant differences; *: p<0.5; **: p<0.05; ***: p<0.01.

FIG. 4 is a schematic representation of fractionation over time (one fraction collected every 3 minutes) of 160° C. and 200° C. subcritical water extracts from three onion varieties. FIG. 4a and FIG. 4b represent 160° C. and 200° C. subcritical water extracts from purple onions (POnion), respectively; FIG. 4c and FIG. 4d represent 160° C. and 200° C. subcritical water extracts from yellow onions (YOnion), respectively; FIG. 4e and FIG. 4f represent 160° C. and 200° C. subcritical water extracts from white onions (WOnion), respectively.

FIG. 5 shows effects of different HPLC fractions (based on time) of 160° C. and 200° C. subcritical water extracts from three onion varieties on the proliferation of human hair dermal papilla cells (HHDPC). FIG. 5a and FIG. 5b represent 160° C. and 200° C. subcritical water extracts from purple onions (POnion), respectively; FIG. 5c and FIG. 5d represent 160° C. and 200° C. subcritical water extracts from yellow onions (YOnion), respectively; FIG. 5e and FIG. 5f represent 160° C. and 200° C. subcritical water extracts from white onions (WOnion), respectively. Water (H2O) and dimethyl sulfoxide (DMSO) serve as control groups. Asterisks (*) indicate significant differences; *: p<0.5; **: p<0.05; ***: p<0.01.

FIG. 6 shows the HPLC fingerprint profiles of extracts obtained from three onion varieties using different extraction methods. FIG. 6a represents the HPLC fingerprint profiles of various extracts obtained from purple onions (POnion) using different extraction methods; FIG. 6b represents the HPLC fingerprint profiles of various extracts obtained from yellow onions (YOnion) using different extraction methods; FIG. 6c represents the HPLC fingerprint profiles of various extracts obtained from white onions (WOnion) using different extraction methods. Signal number: No. 1 represents 200° C. subcritical water extracts, No. 2 represents 160° C. subcritical water extracts, No. 3 represents 150° C. subcritical water extracts, No. 4 represents 140° C. subcritical water extracts, No. 5 represents 130° C. subcritical water extracts, No. 6 represents 120° C. subcritical water extracts, No. 7 represents fresh onion juice extraction, No. 8 represents 95% Ethanol (EtOH) extraction, No. 9 represents ethyl acetate (EA) extraction, and No. 10 represents hexanes (H) extraction.

FIG. 7 shows the HPLC fingerprint profiles of 200° C. subcritical water extracts compared with quercetin. Signal (A) represents 200° C. subcritical water extracts of white onion (WOnion); Signal (B) represents 200° C. subcritical water extracts of purple onion (POnion); Signal (C) represents 200° C. subcritical water extracts of yellow onion (YOnion); and Signal (D) represents quercetin.

### DETAILED DESCRIPTION OF THE INVENTION

The present invention relates to an extract from a natural plant and its extraction method, wherein said extract demonstrates efficacy in promoting hair growth.

The present invnetion provides a method for preparing an onion extract, comprising obtaining an onion sample and subjecting said onion sample to extraction using a subcritical water extraction method, with an extraction temperature ranging from 120° C. to 200° C., an extraction pressure ranging from 1300 to 1700 Psi, and an extraction time ranging from 5 to 45 minutes, resulting in an onion extract.

Preferably, the extraction temperature is between 140° C. and 200° C.

Preferably, the extraction time is 15 minutes.

Furthermore, the present invention also includes a High-Performance Liquid Chromatography (HPLC) fractionation step to obtain at least one fraction of the onion extract, which is performed based on signal intensity or fractionation time.

When fractionation is performed based on signal intensity, it is performed based on the major peak, and the onion extract can be divided into three fractions: Fraction 1 (pre-major peak fraction), Fraction 2 (major peak fraction), and Fraction 3 (post-major peak fraction).

When fractionation is performed based on fractionation time, it is performed every 3 minutes, resulting in a total of five fractions of the onion extract, namely Fraction A, Fraction B, Fraction C, Fraction D, and Fraction E.

Preferably, the fractions of the onion extract of the present invention involve taking Fraction B, Fraction C, Fraction D, and Fraction E; more preferably, the fractions of the onion extract of the present invention involve taking Fraction C, Fraction D, and Fraction E.

In the present invention, the onion sample is selected from onion varieties including yellow onions, purple onions, or white onions.

Preferably, the onion variety used in the present invention is yellow onions or purple onions.

The present invention also provides an onion extract obtained by the method of the present invention.

The present invention further provides a method for promoting hair growth, comprising administering an onion extract to a subject in need thereof.

The subject in the present invention, wherein the subject is human or mammal.

The subject in the present invention, wherein the subject is suffering from alopecia caused by the reasons of pressure, disease or treatment, or the subject is less of hair volume inborn.

Preferably, the onion extract can be further combined with a pharmaceutically acceptable carrier to be formulated into a medicament, or the onion extract can be used as a major ingredient to be formulated to a cosmetic.

The medicament of the present invention comprises pharmaceutically acceptable excipients, particularly those that may further comprise predetermined solvents or oils, pH adjusters, and may further comprise dispersants if required. Examples of solvents used include, but are not limited to, water, ethanol, isopropanol, 1,3-butylene glycol, propylene glycol, glycerin, and the like. Examples of oils used are selected from the group consisting of corn oil, sesame oil, flaxseed oil, cottonseed oil, soybean oil, peanut oil, mono-glycerides, di-glycerides, tri-glycerides, mineral oil, deep-sea shark liver oil (squalene), jojoba oil, olive oil, evening primrose oil, borage oil, grape seed oil, coconut oil, sunflower seed oil, shea butter, and any combinations thereof, but not limited thereto.

The solvents and the oils may be used individually or in any combination thereof.

Examples of dispersants that are beneficial to the present invention may include but not limited to lecithin, organic mono-glycerides, sorbitan fatty acid esters, polyoxyethylene fatty acid esters, and sorbitan stearate. These ingredients may also be used individually or in any combinations thereof.

In the present invention, the medicament of the present invention can be formulated into topical preparations. Examples of the topical preparations include but are not limited to creams, ointments, gels, lotions, patches, as well as inhalants, aerosols, suppositories, and the like.

When the medicament is used as a topical preparation, appropriate dermal preparations for external use can be used as the base material. Aqueous solutions, non aqueous solvents, suspensions, emulsions, gels, creams, ointments, and lyophilized preparations all can be employed and sterilized by established methods. Compositions in the form of gels, creams, and ointments can be appropriately prepared according to the form of the composition by using established methods. This is achieved through the addition of well-known softening agents, emulsifiers, thickeners, or other materials already known in the field of technology.

Compositions in the form of gels, for example, can be prepared by adding softening agents such as trimethylolpropane, polyethylene glycol, and glycerin; solvents such as propylene glycol, ethanol, and isocetyl alcohol; and purified water.

### DESCRIPTION OF EMBODIMENTS

The following examples are not intended to limit the scope of the present invention, but are merely intended to implement the present invention as described above and make other objects, features and advantages of the present invention more apparent.

### Methods

### I. Extract Preparation

### 1. Raw Material Extraction

The onion raw materials for the present invention were sourced from three common onion varieties available in the market, namely purple onions (POnion), yellow onions (YOnion), and white onions (WOnion). Among these, purple onions and yellow onions were produced in Taiwan, while white onions were produced in the United States.

The extraction methods are categorized into the following four types:

(1) High-pressure organic solvent extraction:

The onion bulbs were frozen dried and subsequently ground into a powdered form using a grinder, 5 grams of the powder were weighed and placed into the stainless steel chamber of an ASE350 extraction equipment. As extraction solvents, either hexanes (H), ethyl acetate (EA), or 95% ethanol (EtOH) were employed. The extraction pressure was set at 1500 Psi, although there was a fluctuation of ±200 Psi within the chamber during the extraction, resulting in an actual extraction pressure ranging from 1300 to 1700 Psi. The extraction was performed at room temperature for a duration of 5 to 45 minutes to process the samples.

In the present invention, the extraction time under optimal extraction conditions was 15 minutes.

The obtained extract was dried with a pressure reduction concentrator and then freeze-dried to obtain an onion organic solvent extract.

(2) Subcritical water extraction method (SW):

The onion bulbs were frozen dried and subsequently ground into a powdered form using a grinder, 5 grams of the powder were weighed and placed into the stainless steel chamber of an ASE350 extraction equipment. Water was used as the extraction solvent. The extraction pressure was set at 1500 Psi, although there was a fluctuation of ±200 Psi within the chamber during the extraction, resulting in an actual extraction pressure ranging from 1300 to 1700 Psi. The temperature was set between 120° C. and 200° C., and the extraction duration ranged from 5 to 45 minutes for sample extraction.

In the present invention, the optimal extraction conditions for temperature and time were as follows: a temperature range of 160° C. to 200° C. and an extraction time of 15 minutes.

The obtained extract was dried using a freeze dryer to obtain an onion subcritical water extract.

(3) Reflux extraction method (RE):

The onion bulbs were frozen dried and subsequently ground into a powdered form using a grinder, 5 grams of the sample were weighed and placed into a concentration bottle. Water was used as the extraction solvent, and the extraction was carried out by heating with a water bath until the water temperature reached 100° C. for a duration of 30 minutes. Subsequently, the obtained extract was dried using a freeze dryer to obtain a water reflux extract.

(4) Fresh onion juice (Ju):

Fresh onions were cut into chunks, placed in a juicer, and blended until a paste was formed. The resulting onion juice was filtered through gauze to remove any solids, and the filtered onion juice was collected in a 50 mL centrifuge tube. It was then centrifuged at 650×g for 30 minutes in a centrifuge, and the supernatant was used for analysis.

### 2 Active Fraction(s) of Onion Extract Fractionation by High Performance Liquid Chromatography (HPLC)

The present invention employed a Shimazu RP-HPLC system (CBM-20A communications bus module, LC-20AP pump, SPD-M20A photodiode array detector) for the fractionation of onion subcritical water extracts.

Experimental conditions: A Sunnset C18 column (5 μm, 20.0 mm ×250 mm) was used as the stationary phase. The mobile phase was a gradient condition of mixing methanol (MeOH) and water (containing 0.1% formic acid): 0-10 min, 10-100% MeOH, 10-15 min, 100% MeOH. The flow rate was set at 10 mL/min, and the detection wavelength was 280 nm. The extracts were filtered through a 0.45 μm filtration membrane.

In the selection of HPLC fractionation, collection was carried out based on signal intensity and time, respectively.

### 3 HPLC Fingerprint Analysis

The present invention conducted fingerprint analysis of the extracts using the Shimazu LC2040C HPLC system.

The extracts were dissolved in methanol or 50% methanol, filtered through a 0.45 μm filtration membrane, and prepared to a concentration of 1000 ppm. A sample volume of 10 μL was injected each time.

Experimental conditions: A Sunniest PhE column (5 μm, 4.6 mm×250 mm) was used as the stationary phase. The mobile phase was a gradient condition of mixing methanol (MeOH) and water: 0-20 min, 10-100% MeOH, 20-30 min, 100% MeOH. The flow rate was set at 1 mL/min, and the column temperature was set at 38° C.

### II. Pharmacological Test

### 1. Cell Proliferation Assay

Human hair dermal papilla cells (HHDPC) were seeded in 24-well plates at

a density of 2×10³ cells per well and cultured at 37° C. in C-MSCM medium. After 24 hours of incubation, the old C-MSCM medium was aspirated.

Each well was replenished with 400 μL of C-MSCM medium, followed by the addition of either the control group solution or the test extracts. The plates were then incubated in a 37° C. incubator for 48 hours. After incubation, 20 μL of WST-1 was added to each well, and the plates were further incubated at 37° C. for 2 hours. Absorbance readings at 450 nm and 620 nm were measured.

### Results

The combination of three onion varieties with four extraction methods and different solvents yielded a total of 24 extracts, labeled as follows: POnion Ju, POnion Re, POnion H, POnion EA, POnion EtOH, POnion SW120, POnion SW160, POnion SW200, YOnion Ju, YOnion Re, YOnion H, YOnion EA, YOnion EtOH, YOnion SW120, YOnion SW160, YOnion SW200, WOnion Ju, WOnion Re, WOnion H, WOnion EA, WOnion EtOH, WOnion SW120, WOnion SW160, and WOnion SW200, respectively.

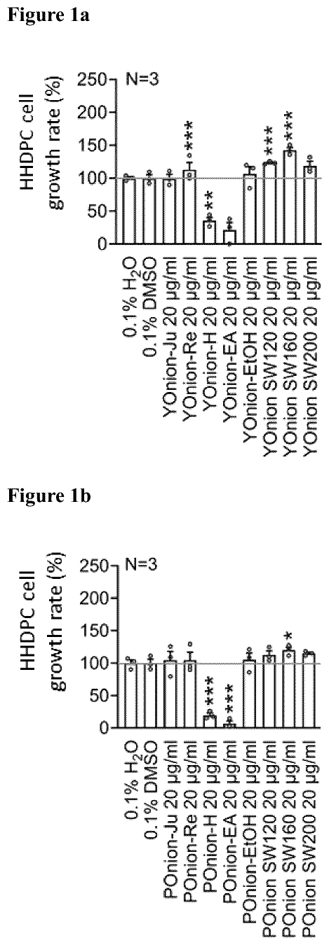

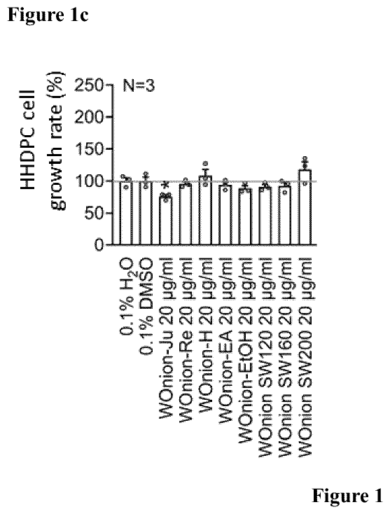

The proliferative effects of various onion extracts at a concentration of 20 μg/mL on human hair dermal papilla cells (HHDPC) were first tested. The results are shown in FIG. 1a to FIG. 1c.

The experimental results indicated that the subcritical water extracts of purple and yellow onions significantly promoted the proliferation of HHDPC, while the hexanes and ethyl acetate extracts of all three types of onions inhibited HHDPC proliferation.

Since the 200° C. subcritical water extracts of all three types of onions showed significant promotion of HHDPC proliferation, the subcritical water extracts of these onions were further stratified by HPLC based on signal intensity.

Fractionation based on signal intensity is performed based on the major peak and divided the subcritical water extracts of each onion into three fractions, labeled Fraction 1 (pre-major peak fraction), Fraction 2 (major peak fraction), and Fraction 3 (post-major peak fraction), respectively.

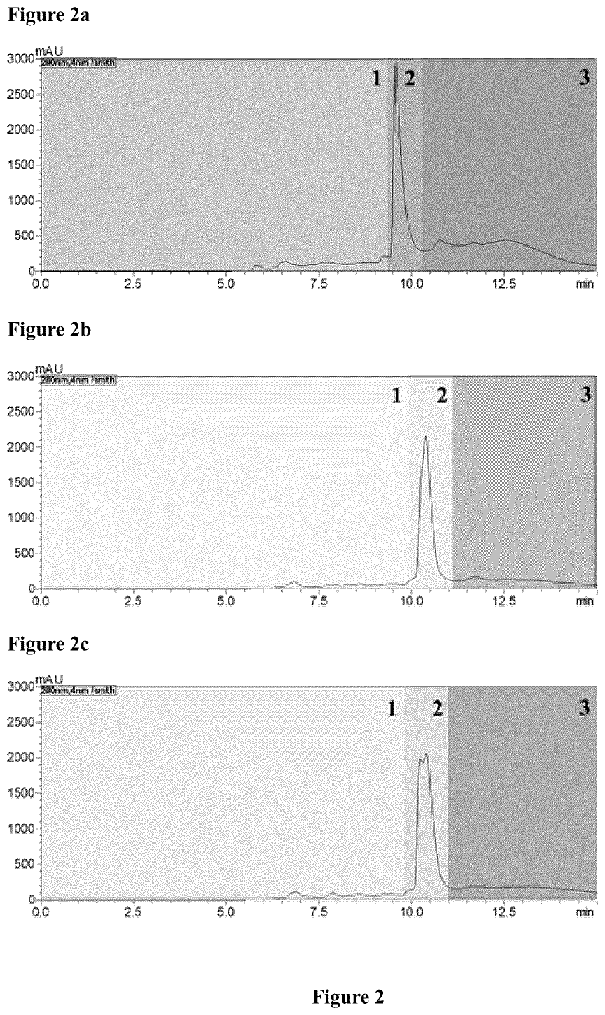

In the present invention, as shown in FIG. 2a to FIG. 2c, a total of 9 fractions were obtained, labeled as POnion SW200-1, POnion SW200-2, POnion SW200-3, YOnion SW200-1, YOnion SW200-2, YOnion SW200-3, WOnion SW200-1, WOnion SW200-2, and WOnion SW200-3, respectively.

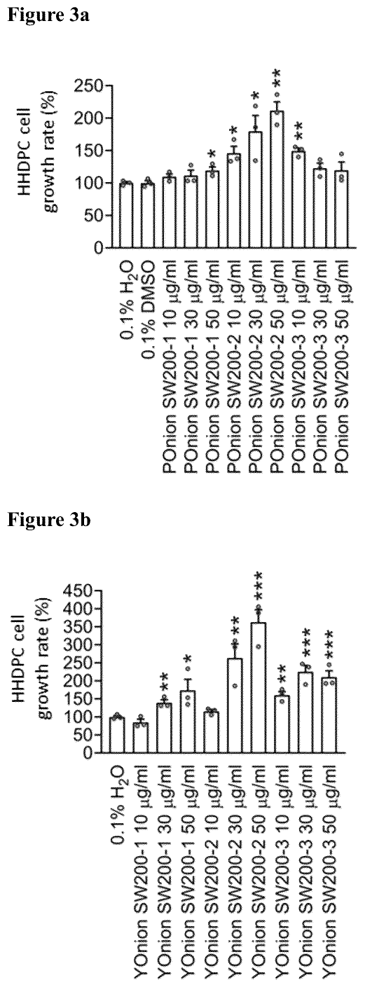

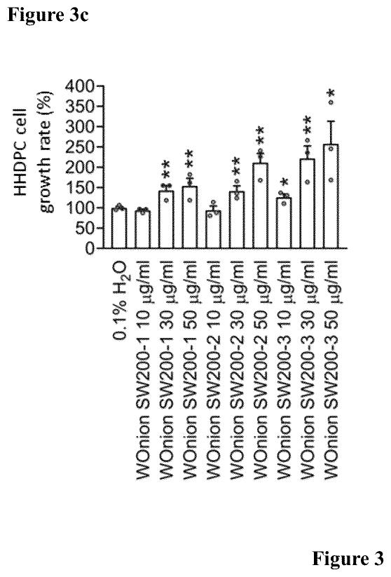

Effects of different fractions of subcritical water extracts from various onions on the proliferation of human hair dermal papilla cells (HHDPC) were tested, and the results are shown in FIG. 3a to FIG. 3c.

The experimental results demonstrated that POnion SW200-2, YOnion SW200-2, WOnion SW200-2, and WOnion SW200-3 all exhibited growth-promoting effects on HHPDC in a concentration-dependent manner.

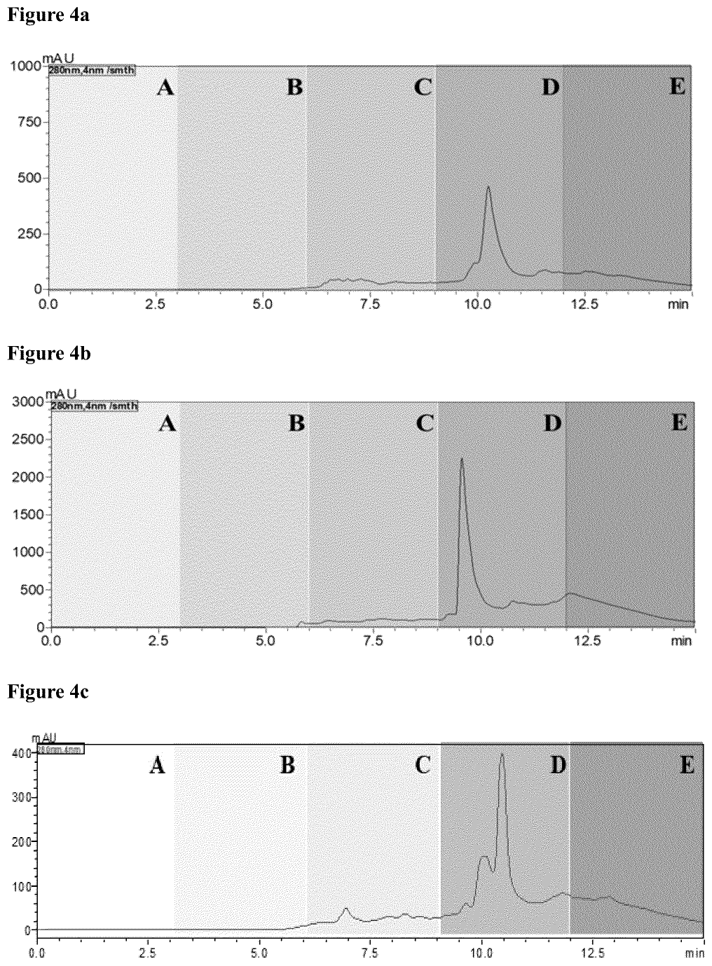

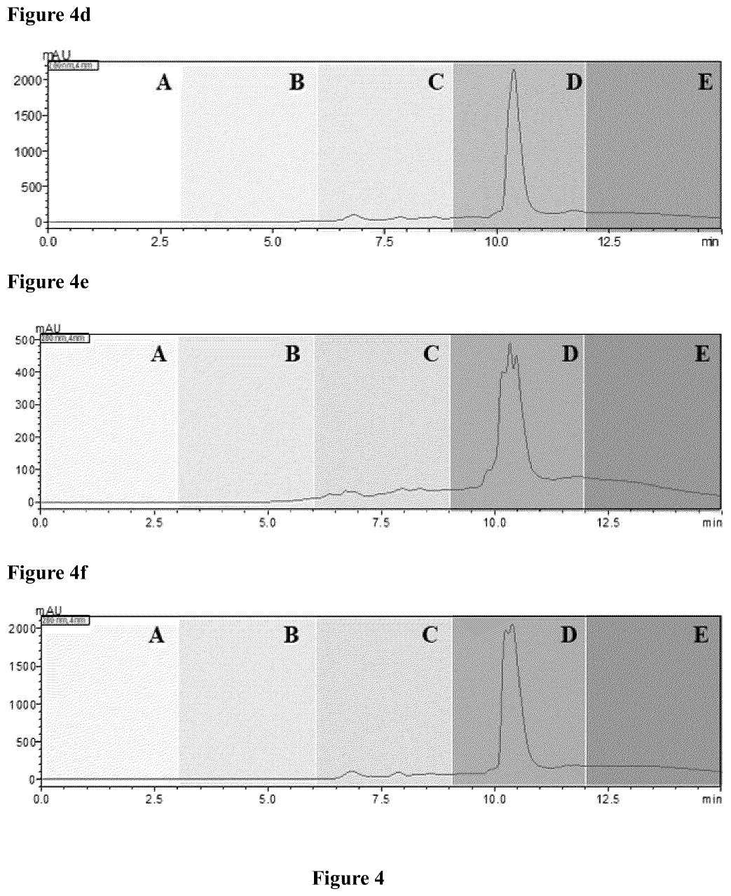

In addition, the 160° C. subcritical water extracts and 200° C. subcritical water extracts of the three onion varieties were stratified based on time, with each fraction collected every 3 minutes, resulting in a total of 15 fractions, as shown in FIG. 4a to FIG. 4f. These fractions were labeled as POnion SW160-A, POnion SW160-B, POnion SW160-C, POnion SW160-D, POnion SW160-E, YOnion SW160-A, YOnion SW160-B, YOnion SW160-C, YOnion SW160-D, YOnion SW160-E, WOnion SW160-A, WOnion SW160-B, WOnion SW160-C, WOnion SW160-D, WOnion SW160-E, POnion SW200-A, POnion SW200-B, POnion SW200-C, POnion SW200-D, POnion SW200-E, YOnion SW200-A, YOnion SW200-B, YOnion SW200-C, YOnion SW200-D, YOnion SW200-E, WOnion SW200-A, WOnion SW200-B, WOnion SW200-C, WOnion SW200-D and WOnion SW200-E.

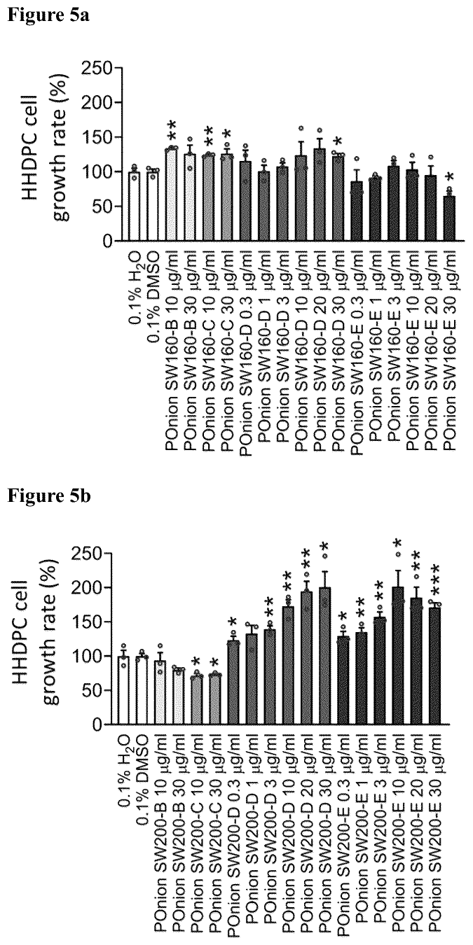

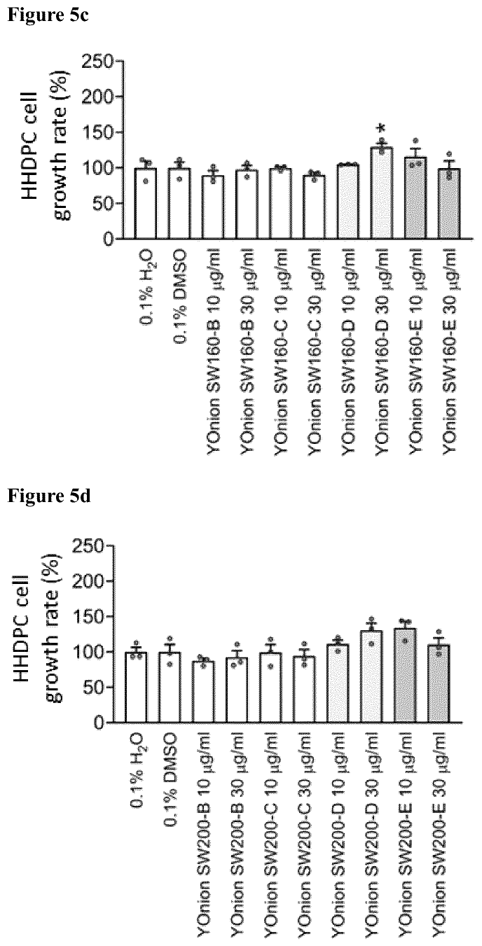

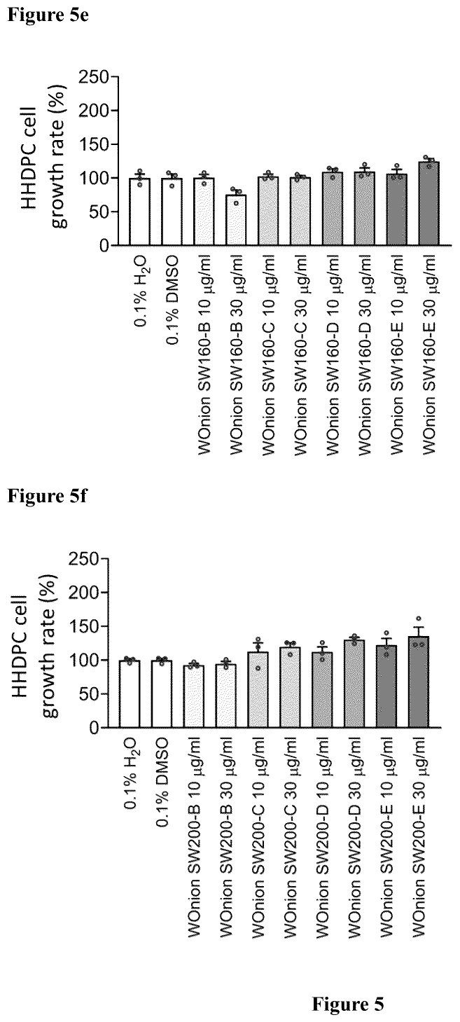

Effects of the above-mentioned different fractions of subcritical water extracts from various onions on the proliferation of human hair dermal papilla cells (HHDPC) were tested, and the results were shown in FIG. 5a to FIG. 5f.

As shown in the experimental results, all the fractions of subcritical water extracts from purple onion extracted at two different temperatures (160° C. and 200° C.) had the effect of promoting the growth of HHPDC, both POnion SW200-D and POnion SW200-E had the better effect of promoting the growth of HHPDC, and a concentration-dependent manner was observed in POnion SW200-D. It is noteworthy that POnion SW200-E achieves its highest activity at a concentration of 10 μg/mL.

In the fractions of subcritical water extracts from yellow onions extracted at two different temperatures (160° C. and 200° C.), it was observed that the fractions of YOnion SW160-D to YOnion SW160-E and YOnion SW200-D to YOnion SW200-E had a stimulating effect on HHDPC proliferation, in which YOnion SW160-D exhibited the best effect at a concentration of 30 μg/mL. However, their effects were all not as significant as those of the POnion SW200-D to POnion SW200-E fractions.

In the fractions of subcritical water extracts from white onions extracted at two different temperatures (160° C. and 200° C.), it was observed that the fractions of WOnion SW200-C to WOnion SW200-E had a stimulating effect on HHDPC proliferation. However, their effects were all not as significant as those of the POnion SW200-D to POnion SW200-E fractions.

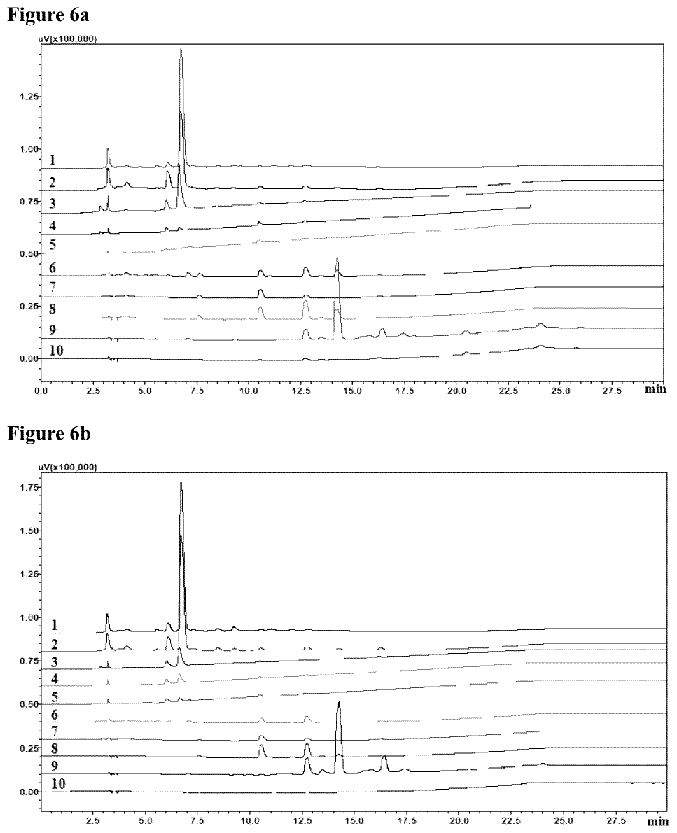

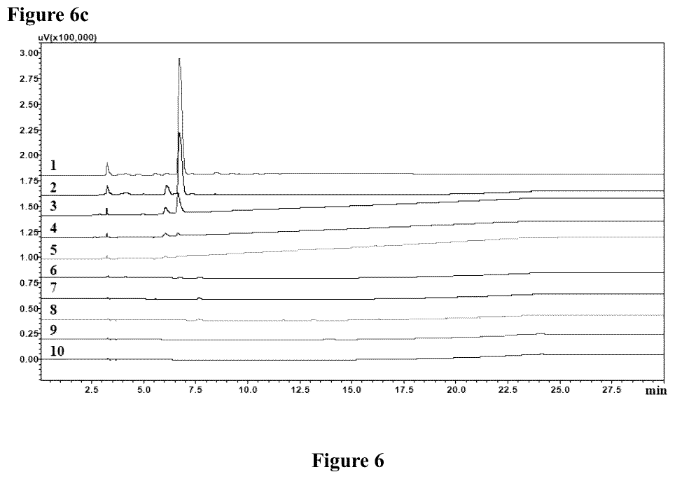

HPLC fingerprint analysis was further performed on various onion subcritical water extracts, and the results were shown in FIG. 6a to FIG. 6c. Signal number: No. 1 represents 200° C. subcritical water extracts, No. 2 represents 160° C. subcritical water extracts, No. 3 represents 150° C. subcritical water extracts, No. 4 represents 140° C. subcritical water extracts, No. 5 represents 130° C. subcritical water extracts, No. 6 represents 120° C. subcritical water extracts, No. 7 represents fresh onion juice extraction, No. 8 represents 95% Ethanol (EtOH) extraction, No. 9 represents ethyl acetate (EA) extraction, and No. 10 represents hexanes (H) extraction.

The fingerprint patterns show that, regardless of the type of onion, the chemical composition signals of the subcritical water extracts generated at 130° C. are distinct from those obtained by other extraction methods. The difference of the chemical signals were significantly observed at the temperature above 140° C., and the strongest chemical signals were exhibited at 160° C. and 200° C.

Previous research has revealed that onion skins are rich in quercetin which possesses strong free radical scavenging abilities. Therefore, the present invention further investigates whether the promotion of hair follicle cell proliferation by 200° C. subcritical water extracts from various onions is attributed to quercetin, as confirmed through HPLC fingerprint profiles.

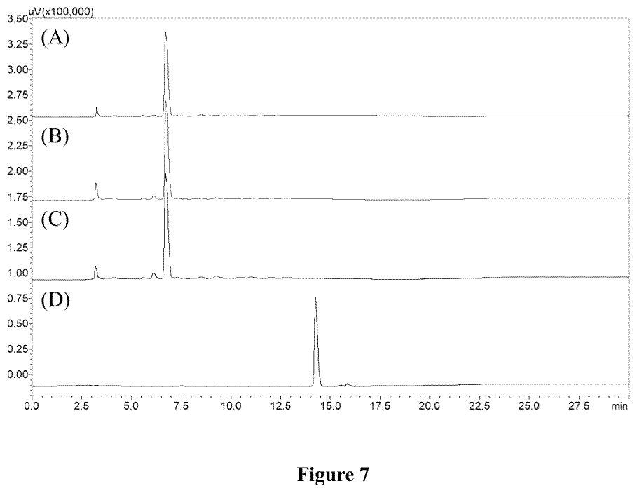

As shown in FIG. 7, signal (A) represents 200° C. subcritical water extracts of white onion (WOnion); Signal (B) represents 200° C. subcritical water extracts of purple onion (POnion); Signal (C) represents 200° C. subcritical water extracts of yellow onion (YOnion); Signal (D) represents quercetin. The result demonstrated that the main compound obtained after the subcritical water extraction process was not quercetin.

In summary, subcritical water extraction of various onion varieties yields extracts with the activity of promoting hair follicle cell proliferation, thus achieving the effect of promoting hair growth. Among these, 200° C. subcritical water extracts exhibit the most significant effects. Among the onion varieties tested, purple onions show the highest activity, followed by yellow onions, with white onions demonstrating the most minor activity.

In order to enable person having ordinary skills in the art to understand the methods of making and using this technology, it has been described and exemplified in detail. However, various variations, modifications, or improvements should be considered to fall within the spirit and scope of the present invention.

Person having ordinary skills in the art can easily understand and achieve the objectives of the present invention, and obtain the previously mentioned results and advantages. The sources of physiological signals, weighting values, or gene sets used in the present invention are provided as examples and not as limitations on the scope of the invention. Modifications or other applications that may arise from those skilled in the art when making or using of this technology are all encompassed within the spirit of the present invention and are limited by the scope of the claims.

What is claimed is:

1. A method for preparing an onion extract, comprising:
step (1): providing an onion sample; and
step (2): extracting the onion sample by a subcritical water extraction method to obtain an onion extract, wherein the extraction temperature ranges from 120° C. to 200° C., the extraction pressure ranges from 1300 to 1700 Psi, and the extraction time ranges from 5 to 45 minutes.

2. The method of claim 1, wherein the method further comprises step (3):
further subjecting the onion extract to high-performance liquid chromatography (HPLC) fractionation to obtained at least one onion extraction fraction, wherein the fractionation is based on signal intensity or fractionation time.

3. The method of claim 2, when the HPLC fractionation is based on signal intensity, three fractions are obtained, namely Fraction 1, Fraction 2, and Fraction 3.

4. The method of claim 2, when the HPLC fractionation is based on fractionation time, it is performed every 3 minutes, resulting in a total of 5 fractions, namely Fraction A, Fraction B, Fraction C, Fraction D, and Fraction E, wherein Fraction B, Fraction C, Fraction D, and Fraction E are used, respectively.

5. The method of claim 4, wherein Fraction C, Fraction D, and Fraction E are used, respectively.

6. The method of claim 1, wherein the extraction temperature ranges from 140° C. to 200° C.

7. The method of claim 1, wherein the extraction time is 15 minutes.

8. The method of claim 1, wherein the onion sample is selected from yellow onion, purple onion, or white onion.

9. The method of claim 1, wherein the onion sample is selected from yellow onion or purple onion.

10. An onion extract prepared by the method of claim 1.

11. A method for promoting hair growth, comprising administering the onion extract of claim 10 to a subject in need thereof.
